(19) United States
(12) Patent Application Publication
(10) Pub. No.: US 2022/0133521 A1
(43) Pub. Date: May 5, 2022

(54) Head and Neck Stabilizing Apparatus

(71) Applicant: Bradford Lee Bentley, Seattle, WA (US)

(72) Inventor: Bradford Lee Bentley, Seattle, WA (US)

(21) Appl. No.: 17/086,351

(22) Filed: Oct. 31, 2020

(51) Int. Cl. A61F 5/055; A61F 5/37

(52) U.S. Cl. CPC A61F 5/055; A61F 5/3769

(57) ABSTRACT

A physical therapeutic apparatus that has a table-stable base, upon which is mounted a fulcrum pivot point, and an occipital cradle engaged upon the pivot point for selectable and lockable angular motion about the pivot point. The apparatus also has an adjustable chin rest, the chin rest having left and right arms, each arm adjustable for length, each arm adjustably mounted for lateral motion at opposite ends of a pivot point, each arm terminating in releasably connectable ends. An optional dorsal wedge is engaged upon the occipital cradle.

### TECHNICAL FIELD

This disclosure relates to head and neck stabilizing devices in an examination or therapeutic setting; more particularly, it relates to an apparatus for use in physical therapy and other medical settings; and in particular for a head and neck stabilization apparatus for use in physical therapy patient evaluation and treatment.

### BACKGROUND

Physical Therapists and other health care providers devote considerable thought and skill in setting and maintaining a therapeutically optimized position for a patient's head and neck while training the muscles of stabilization and mobilizing the cervical spine.

Much of this is done in accordance with a preliminary examination and accomplished with the therapist's skilled intervention. In some cases, support devices are used to assist with the examination or therapeutic mobilizations/manipulations, or both. Some commonly used device aids are special ‘ contour’ pillows, preformed neck rolls, and sometimes just a simple rolled towel.

At least one aim of such positioning during examination and therapy phases is to flex the neck to a particular curvature that is optimized for the individual patients, and then to hold or maintain that neck curvature during a particular therapeutic mobilization/manipulation or treatment.

A number of devices related to the human head and neck have been disclosed. For instance, there are disclosures of apparati that are attached to the head, and some of these devices have members that fit on or behind the head. Some of them also have members which engage the chin.

For instance, the Ballert Oxford Collar prosthetic device (2011) is a frame type cervical orthotic (www.ballert.com). It has a wire frame covered with foam for comfort and a spring action mandibular piece. It also has a leather adjustable strap which fits behind the neck. The stated purpose of the device is to help balance a patient's head on her shoulders and prevent the neck from flexing (chin going down on chest). It is only worn for certain tasks such as reading or watching television or for other times when patients become fatigued.

Sham Inc. Discloses a Jaw Elevation Device (JED)(www.sharn.com). It is an “externally applied, non-invasive device that maintains an open airway in situations when breathing is in danger of being compromised.”

Hardcastle (U.S. patent Ser. No. 10/226,374 Mar. 12, 2019) discloses a cervical brace variant of the Ballert collar.

Lanier (U.S. Pat. No. 5,524,639) discloses a jaw support variant of the Sham JED.

Farnum (U.S. Pat. No. 8,469,032) disclosed an airway positioning device like the Sharn JED.

Day (U.S. Pat. No. 4,545,572) discloses a wall-mounted head holding surgical device.

Kostich (U.S. Pat. No. 5,081,665) discloses a device for holding, positioning and immobilizing a human head in prone or supine positions during medical treatments or examinations. The device supports frontal and mandibular portions of the skull with two mounted arcuate supports.

Tranquil Neck Support (2017) An adjustable decline device to support resting neck posture and extension has height adjustable neck rest and height adjustable head rest on a base. (https://stacksocial.com/sales/tranquil-2-0)

It is to be noted that, while such devices do engage the head of a patient (and the cervical orthotic examples have a relation to the neck as well), none of these devices provide, or attempt to provide, the range of motion and adjustable members for setting and maintaining an optimal head/neck position for therapeutic muscle training of the deep cervical muscles of stabilization and for mobilization/manipulation of the cervical spine.

What is needed is a physical therapy apparatus that has an occipital cradle on a base with a fulcrum pivot point proximate to the individual's OA joint (the atlanto-occipital joint that provides articulation between the atlas and the occipital bone) together with adjustable and releasably connectable chin rest members and an optional dorsal wedge (for suboccipital muscle release) such that a gentle chin nod into flexion produces a ‘see saw’ effect, a stretch of the suboccipital area, and recruitment of the longus colli, longus capitis, and rectus capitis anterior muscles (the longus colli are on the anterior surface of the vertebral column, between the atlas and third thoracic vertebra, while the longus capitis and rectus capitis anterior are located anteriorly between the underside of the cranium and the upper cervical vertebrae. Together they produce craniocervical flexion via a chin nod motion and are involved with stabilizing the head and neck).

### DISCLOSURE

Such a device is disclosed, and represents a system allowing, and uniquely capable of providing, such a chin nod ‘see saw’ effect, including the stretch of the suboccipital area and the recruitment of the longus colli, longus capitis, and rectus capitis anterior muscles.

A physical therapeutic apparatus with a table-stable base is disclosed. A fulcrum pivot point is mounted on the base and an occipital cradle is engaged upon the pivot point for selectable and lockable angular motion about the pivot point. An optional dorsal wedge is provided on the cradle for increased suboccipital release/stretch. The apparatus also advantageously has an adjustable chin rest, the chin rest having left and right arms, each arm adjustable for length, each arm adjustably mounted for lateral motion at opposite ends of a pivot point, each arm terminating in releasably connectable ends.

The disclosed apparatus has its occipital cradle engaged upon the base via a fulcrum pivot point that is proximate to the patient's OA joint. The adjustable and connectable chin rest members are engaged such that a gentle chin nod into flexion produces a ‘see saw’ effect, a stretch of the suboccipital area and recruitment of the longus colli, etc.

A novel apparatus having particular application for physical therapy professionals and patients (as well as therapeutically similarly situated medical professions and their patients) is disclosed. The apparatus sits on a base which is advantageously table or floor stable, which is to say that when the base is placed upon a table, table-like surface or level floor, it is stable in the various degrees of possible motion or deflection. In other words, while it is capable of sliding on the table unless secured in position, it is resistant to tipping or deflecting in any direction and to any significant extent. The base may be made of any suitable material and most any shape, consistent with the stability objectives summarized here.

Mounted upon the base, such as by integrated attachment or integrated manufacture, or by releasably lockable engagement, such as within a slot or aperture let into the base, is a pivot point member providing a fulcrum for releasably lockable movement of the occipital cradle in at least one range of motion and or one degree of freedom of motion and or around at least one axis of rotation. Desirably, the pivot point member provides a fulcrum for releasably lockable movement of the occipital cradle in a plurality of ranges of motion and or multiple degrees of freedom of motion and or around any of a plurality of axes of rotation. Advantageously, the pivot point member may be a ball and socket assembly, one part of the assembly engaged on or within the base and the other part engaged on or within the occipital cradle part of the apparatus, so that the cradle is selectably tiltable and rotatable with respect to the base. Desirably, the ball and socket assembly includes conventional releasable position locking means. Other pivot point assemblies can be made to serve as well, as will be appreciated by those skilled in the mechanical assembly arts.

The occipital cradle member is appropriately shaped for receiving the back of the head for a range of head sizes, and thus the patient's head, when either lying in the cradle and maintaining its position by weight or by optional securing means, is engaged upon the cradle which is in turn engaged upon the pivot point assembly for selectable and lockable angular motion about the pivot point. The shape of the occipital cradle and the relative positioning (advantageously adjustable in this respect) of the engaged pivot point beneath the cradle is such as to provide for the patient's OA joint to lie on the cradle directly above the pivot point. In other words, the pivot point member is releasably movable beneath the cradle for optimizing position of the pivot point relative to a patient OA joint.

Advantageously, a suboccipital protuberance (such as a wedge) is temporarily or permanently engaged upon an upper surface of the occipital cradle for engagement of the dorsal surface of a patient's head. Whether temporary or permanent engaged, the dorsal wedge is desirably adjustable longitudinally for advantageous adjustment of the wedge to suit the particular patient head and the desired amount of additional ‘see saw’ tilt of the head during therapeutic use of the apparatus. The dorsal wedge is also desirably adjustable both in and out, with respect to the occipital cradle, so that it has a range of in and out adjusted motion to put the dorsal touching surface of the wedge below the surface of the cradle, or to extend it fully above the cradle for maximum additional ‘see saw’ tilt of the patient head (made longer or shorter from projecting up out of the cradle to being all the way down and relatively flush with the cradle).

In addition to ‘see saw’ tilt motion, the wedge and its adjustments are believed also to provide an adjustable range of stretch of the suboccipital area of the patient's head/neck and an adjustable amount of recruitment of the longus colli, etc. Conventional means of such adjustments of the dorsal wedge in any of the directions or extents disclosed will be within the scope of persons skilled in the mechanical assembly arts.

An adjustable chin rest assembly is mounted or otherwise suitably engaged upon or along the occipital cradle and tilts, rotates or otherwise moves with the cradle as it moves upon the pivot point assembly. The chin rest assembly desirably has left and right arms, each arm independently adjustable for length, each arm swingingly and adjustably engaged at opposite sides of its mount or point of engagement for independent lateral and swinging or rotational motion at the point of engagement with the cradle, each arm terminating in releasably connectable ends. The releasable ends may be any of many conventional temporarily lockable but releasable means to secure the two arms together as a temporary and unlockable chin rest. On such means is a pair of appropriately and oppositely poled magnets, one part of the pair mounted in each of the connectable ends of the two arms.

In some embodiments of the disclosed apparatus, it is advantageous to provide releasable locking means to move the pivot point assembly (fulcrum) either closer to the chin rest mounting point, or farther away from it; and or to move the chin rest occipital cradle mounting closer to the pivot point or farther away from it.

### BRIEF DESCRIPTION OF THE DRAWINGS

FIG. 1 is a side elevation of a conventional head-securing means.

FIG. 2 is a side elevation of an aspect of the disclosed apparatus.

FIG. 3 is a perspective schematic view of an aspect of the disclosed apparatus.

FIG. 4 is a perspective schematic view of an aspect of the disclosed apparatus.

FIG. 5 is a schematic plan view of an aspect of the disclosed apparatus.

### DETAILED DESCRIPTION

Turning now to the drawings, the disclosed apparatus is described by reference to the numerals of the drawing figures wherein like numbers indicate like parts.

FIG. 1 shows a conventional head-securing means. The head is simply secured by a strap to a soft base, with the neck resting on a conventional neck support.

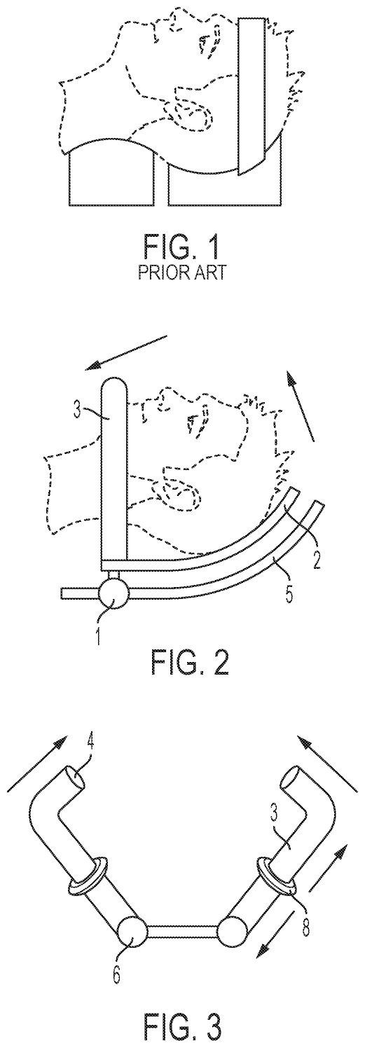

FIG. 2 shows a patient head engaged upon occipital cradle 2 and also engaged within closed adjustable chin rest arms 3. Cradle 2 is engaged upon fulcrum pivot point 1 for relative motion (shown for example by the directions of the arrows in the figure) of head and cradle about pivot point 1 relative to base 5.

FIG. 3 is a detail of open and adjustable chin rest arms 3. Each arm 3 is adjustable in length at adjustment clamp 8 (in the directions shown by the arrows near the clamp in the figure). Each arm 3 has one half of chin rest locking assembly 4. Each arm 3 is also swingable laterally (away from each other and from chin rest articulation joint 6 (in directions approximated schematically by the slanted arrows in the figure), as well as rotatably about chin rest articulation joint 6 (in directions in and out of the plane of the figure).

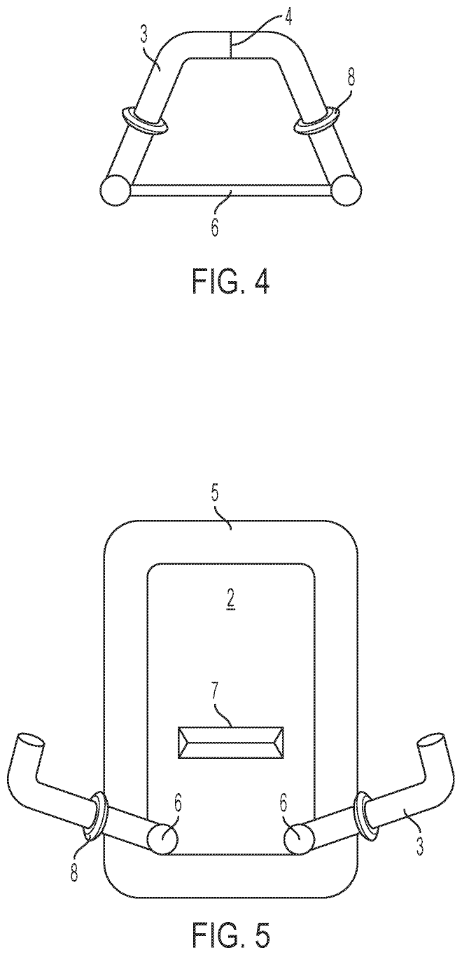

FIG. 4 is a detail of closed and locked chin rest arms 3 to form the chin rest. Each arm 3 has been adjusted in length at adjustment clamp 8; each arm 3 is shown locked to the other by respective halves of chin rest locking assembly 4. Each arm 3 is shown fixed with respect to chin rest articulation joint 6.

FIG. 5 is a plan view schematic of the disclosed apparatus. Base 5 holds occipital cradle 2 (by means of pivot point assembly 1 hidden and not shown). Dorsal wedge 7 is shown interengaged with cradle 2 (adjusting means not shown). Open and adjustable chin rest arms 3 and their adjustment clamps 8 are schematically shown interengaged upon (combined or independent) chin rest articulation joints 6 which in turn are interengaged upon cradle 2.

With regard to systems and components above referred to, but not otherwise specified or described in detail herein, the workings and specifications of such systems and components and the manner in which they may be made or assembled or used, both cooperatively with each other and with the other elements of the invention described herein to effect the purposes herein disclosed, are all believed to be well within the knowledge of those skilled in the art. No concerted attempt to repeat here what is generally known to the artisan has therefore been made.

In compliance with the statute, the invention has been described in language more or less specific as to structural features. It is to be understood, however, that the invention is not limited to the specific features shown, since the means and construction shown comprise preferred forms of putting the invention into effect. The invention is, therefore, claimed in any of its forms or modifications within the legitimate and valid scope of the appended claims, appropriately interpreted in accordance with the doctrine of equivalents.

I claim:

1. A physical therapeutic apparatus comprising:
a) a base, upon which is mounted a fulcrum pivot point member;
b) an occipital cradle engaged upon the pivot point for selectable and lockable angular motion about the pivot point.

2. The apparatus of claim 1 further comprising an adjustable chin rest on a mount, the chin rest comprising:
left and right arms, each arm adjustable for length, each arm adjustably engaged upon the mount for lateral motion at opposite ends of a pivot point, each arm terminating in releasably connectable ends.

3. The apparatus of claim 2 wherein the respective releasable ends are appropriately and oppositely poled magnets.

4. The apparatus of claim 1 further comprising a suboccipital wedge engaged upon the occipital cradle.

5. The apparatus of claim 4 wherein the wedge is adjustably engaged upon the occipital cradle for optimizing one or more of the group of therapeutic effects, the group consisting of a ‘see saw’ effect, a stretch of suboccipital area, and a recruiting of at least one of the group of muscles, the group consisting of the longus colli, the longus capitis, and the rectus capitis anterior.

6. The apparatus of claim 4 wherein the wedge is adjustably engaged upon the occipital cradle and can be adjustably made longer or shorter from projecting up out of the cradle to being all the way down and relatively flush with the cradle.

7. The apparatus of claim 1 wherein the pivot point member is releasably movable beneath the cradle for optimizing position of the pivot point relative to a patient OA joint.

8. The apparatus of claim 2 wherein the pivot point member is a releasably lockable roller ball member adjustable for movement closer to the chin rest or farther away from it.

9. The apparatus of claim 2 wherein the chin rest mount is releasably adjustable upon the base for movement closer to the pivot point or farther away from it.

10. An adjustable chin rest mounted on a base, the chin rest comprising:
left and right arms, each arm adjustable for length, each arm adjustably mounted for lateral motion at opposite ends of an arm pivot point, each arm terminating in releasably connectable ends.

11. The apparatus of claim 10 wherein the respective releasable ends are appropriately and oppositely poled magnets.

12. A physical therapeutic apparatus comprising:
a) a base, upon which is mounted a fulcrum pivot point member;
b) an occipital cradle engaged upon the pivot point for selectable and lockable angular motion about the pivot point;
c) an adjustable chin rest on a mount, the chin rest comprising left and right arms, each arm adjustable for length, each arm adjustably engaged upon the mount for lateral motion at opposite ends of a pivot point, each arm terminating in releasably connectable ends;
d) a suboccipital wedge engaged upon the occipital cradle.

13. The apparatus of claim 12 wherein the wedge is adjustably engaged upon the occipital cradle for optimizing one or more of the group of therapeutic effects, the group consisting of a ‘see saw’ effect, a stretch of suboccipital area, and a recruiting of at least one of the group of muscles, the group consisting of the longus colli, the longus capitis, and the rectus capitis anterior.

14. The apparatus of claim 13 wherein the therapeutic effect is a ‘see saw’ effect.

15. The apparatus of claim 12 wherein the wedge adjustably engaged upon the occipital cradle can be adjustably made longer or shorter from projecting up out of the cradle to being all the way down and relatively flush with the cradle.

16. The apparatus of claim 12 wherein the pivot point member is releasably movable beneath the cradle for optimizing position of the pivot point relative to a patient OA joint.

17. The apparatus of claim 12 wherein the pivot point member is a releasably lockable roller ball member adjustable for movement closer to the chin rest or farther away from it.

18. The apparatus of claim 12 wherein the chin rest mount is releasably adjustable upon the base for movement closer to the pivot point or farther away from it.
